(19) United States
(12) Patent Application Publication
(10) Pub. No.: US 2025/0295780 A1
(43) Pub. Date: Sep. 25, 2025

(54) REACTIVE OXYGEN SPECIES-RESPONSIVE DRUG DELIVERY PARTICLES, WOUND HEALING METHOD USING REACTIVE OXYGEN SPECIES-RESPONSIVE DRUG DELIVERY PARTICLES AND PHOTOBIOMODULATION, AND DEVICE FOR WOUND HEALING

(71) Applicants: KOREA ADVANCED INSTITUTE OF SCIENCE AND TECHNOLOGY, Daejeon (KR); KOREA INSTITUTE OF CERAMIC ENGINEERING AND TECHNOLOGY, Gyeongsangnam-do (KR)

(72) Inventors: Kyung Cheol CHOI, Daejeon (KR); Hyejeong YEON, Daejeon (KR); Daekyung SUNG, Sejong-si (KR); Sangwoo KIM, Gyeonggi-do (KR); Sohyeon YU, Chungcheongbuk-do (KR)

(21) Appl. No.: 18/918,549

(22) Filed: Oct. 17, 2024

(30) Foreign Application Priority Data
Mar. 19, 2024 (KR) 10-2024-0038101

(51) Int. Cl. A61K 41/00; A61K 9/00; A61K 9/16; A61K 36/23

(52) U.S. Cl. CPC A61K 41/0057; A61K 9/0014; A61K 9/1635; A61K 36/23

(57) ABSTRACT

Disclosed are reactive oxygen species-responsive drug delivery particles including: a ferrocene particle containing a polymer containing ferrocene; and a reactive oxygen species-scavenging drug loaded in the ferrocene particle, wherein the ferrocene particle contains ferrocenylmethyl methacrylate, polyethylene glycol methacrylate bound to the ferrocenylmethyl methacrylate, and methacrylic acid bound to the polyethylene glycol methacrylate, the ferrocenylmethyl methacrylate, the polyethylene glycol methacrylate, and the methacrylic acid having a molar ratio of 1:1:1 to 1:5:10.

### CROSS-REFERENCE TO RELATED APPLICATION(S)

This application is based on and claims priority under 35 U.S.C. 119 to Korean Patent Application No. 10-2024-0038101, filed on Mar. 19, 2024, in the Korean Intellectual Property Office, the disclosure of which is herein incorporated by reference in its entirety.

### FIELD

The present disclosure relates to reactive oxygen species-responsive drug delivery particles, a wound healing method using reactive oxygen species-responsive drug delivery particles and photobiomodulation, and a device for wound healing and, more specifically, to reactive oxygen species-responsive drug delivery particles releasing a reactive oxygen species-scavenging drug in response to reactive oxygen species generated by photobiomodulation, a wound healing method using the reactive oxygen species-responsive drug delivery particles and photobiomodulation, and a device for wound healing.

### BACKGROUND

Wearable medical devices provide technology capable of diagnosing physiological conditions and treating diseases without any time and space limitations. Particularly, light-based medical technology has the advantages of noninvasiveness, nontoxicity, and effectiveness and is utilized in photobiomodulation (PBM), photodynamic therapy (PDT), pulse oximetry, and others.

In the medical field, phototherapy is receiving attention as a method for health promotion or treatment. Phototherapy is a technique that allows light to be absorbed into the skin of the human body to activate, regenerate, or destroy specific tissues within the skin. Especially, the red light region is generally known to be effective in wound healing and cell proliferation.

Photobiomodulation, a chemotherapeutic approach using light, can treat various types of diseases by promoting the cellular metabolic activity through only low-level light irradiation. Previously, phototherapies using lasers and LEDs were possible only in restricted environments, such as medical facilities, resulting in limitations, such as low convenience and inadaptability.

In photodynamic therapy, the rate of reactive oxygen species (ROS) generated increases in proportion to light energy and light intensity, with higher reactive oxygen species generation leading to greater treatment efficiency. In photodynamic therapy, a photosensitizer activated by light converts surrounding oxygen into reactive oxygen species, which then attack and kill cancer cells, thereby enhancing the efficiency of cancer treatment.

On the other hand, in photobiomodulation, an increased amount or concentration of reactive oxygen species within cells enhances a therapeutic effect, but abnormality occurs in that the therapeutic effect diminishes as the amount of reactive oxygen species exceeds a predetermined amount.

### SUMMARY

An aspect of the present disclosure is to provide reactive oxygen species-responsive drug delivery particles, which enhance a wound healing effect by releasing a reactive oxygen species-scavenging drug in response to reactive oxygen species generated by photobiomodulation, a wound healing method using reactive oxygen species-responsive drug delivery particles and photobiomodulation, and a device for wound healing.

In accordance with an aspect of the present disclosure, there are provided reactive oxygen species-responsive drug delivery particles including: a ferrocene particle containing a polymer containing ferrocene; and a reactive oxygen species-scavenging drug loaded in the ferrocene particle.

In an embodiment of the present disclosure, the ferrocene particle may contain ferrocenylmethyl methacrylate, polyethylene glycol methacrylate bound to the ferrocenylmethyl methacrylate, and methacrylic acid bound to the polyethylene glycol methacrylate, the ferrocenylmethyl methacrylate, the polyethylene glycol methacrylate, and the methacrylic acid having a molar ratio of 1:1:1 to 1:5:10.

In an embodiment of the present disclosure, the ferrocene particle may be represented by Chemical Formula 1 below:

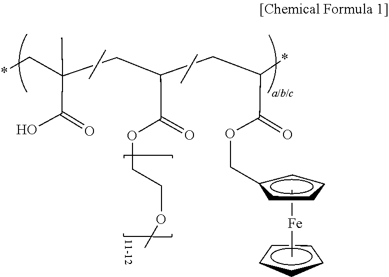

In an embodiment of the present disclosure, the reactive oxygen species-scavenging drug may contain a Centella asiatica component.

In an embodiment of the present disclosure, the ferrocene particle may release the reactive oxygen species-scavenging drug in response to a light with a wavelength of 600 to 700 nm.

In accordance with another aspect of the present disclosure, there is provided a wound healing method, including: preparing reactive oxygen species-responsive drug delivery particles containing a reactive oxygen species-scavenging drug; attaching the reactive oxygen species-responsive drug delivery particles to the human skin; irradiating the human skin with a light with a predetermined wavelength; generating reactive oxygen species by photobiomodulation using the light; releasing the reactive oxygen species-responsive drug loaded in the reactive oxygen species-responsive drug delivery particles by the reactive oxygen species; and scavenging some of the reactive oxygen species by the reactive oxygen species-scavenging drug.

In an embodiment of the present disclosure, the reactive oxygen species-responsive drug delivery particles may include: a ferrocene particle containing ferrocenylmethyl methacrylate, polyethylene glycol methacrylate bound to the ferrocenylmethyl methacrylate, and methacrylic acid bound to the polyethylene glycol methacrylate; and a reactive oxygen species-scavenging drug loaded in the ferrocene particle, the ferrocenylmethyl methacrylate, the ferrocenylmethyl methacrylate, and the polyethylene glycol methacrylate having a molar ratio of 1:1:1 to 1:5:10.

In an embodiment of the present disclosure, the ferrocene particle may be represented by Chemical Formula 1 below:

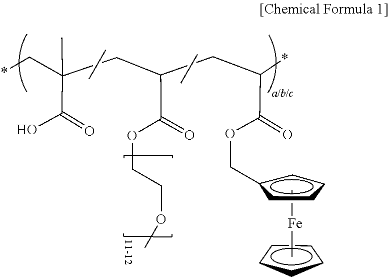

In an embodiment of the present disclosure, the reactive oxygen species-scavenging drug may contain a Centella asiatica component.

In an embodiment of the present disclosure, the ferrocene particle may release the reactive oxygen species-scavenging drug in response to a light with a wavelength of 600 to 700 nm.

In accordance with still another aspect of the present disclosure, there is provided a device for wound healing, including: a band part including a first surface with an adhesive layer formed thereon; a light source part disposed above the first surface to emit a light with a predetermined wavelength; a power supply part disposed above the first surface and electrically connected to the light source part; and reactive oxygen species-responsive drug delivery particles disposed on the first surface, wherein the reactive oxygen species-responsive drug delivery particles include: a ferrocene particle containing a polymer containing ferrocene; and a reactive oxygen species-scavenging drug loaded in the ferrocene particle.

In an embodiment of the present disclosure, the ferrocene particle may contain ferrocenylmethyl methacrylate, polyethylene glycol methacrylate bound to the ferrocenylmethyl methacrylate, and methacrylic acid bound to the polyethylene glycol methacrylate, the ferrocenylmethyl methacrylate, the polyethylene glycol methacrylate, and the methacrylic acid having a molar ratio of 1:1:1 to 1:5:10.

In an embodiment of the present disclosure, the ferrocene particle may be represented by Chemical Formula 1 below:

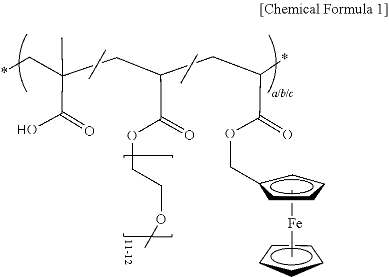

In an embodiment of the present disclosure, the reactive oxygen species-scavenging drug may contain a Centella asiatica component.

In an embodiment of the present disclosure, the reactive oxygen species-responsive drug delivery particles may be applied onto an emission surface of the light source part.

In an embodiment of the present disclosure, the ferrocene particle may release the reactive oxygen species-scavenging drug in response to a light with a wavelength of 600 to 700 nm.

According to the embodiments of the present disclosure, the level of reactive oxygen species within cells can be adjusted to an appropriate amount by scavenging excessive reactive oxygen species generated by photobiomodulation.

Furthermore, according to the embodiments of the present disclosure, the wound healing effect can be enhanced by increasing the cell migration and cell proliferation in a wound site.

### BRIEF DESCRIPTION OF THE DRAWINGS

The above and other aspects, features and advantages of the present disclosure will be more apparent from the following detailed description taken in conjunction with the accompanying drawings.

FIG. 1 shows reactive oxygen species-responsive drug delivery particle according to an embodiment of the present disclosure and the release of a drug from the reactive oxygen species-responsive drug delivery particle.

FIG. 2 is a perspective diagram schematically showing a device for wound healing according to an embodiment of the present disclosure.

FIG. 3 is a flow chart of a treatment method according to an embodiment of the present disclosure.

FIG. 4 schematically shows a treatment method according to an embodiment of the present disclosure.

FIG. 5 shows a treatment procedure by a treatment method according to an embodiment of the present disclosure.

FIG. 6 is a graph showing the generation of reactive oxygen species depending on the light energy density.

FIG. 7 is a graph showing the drug release rate depending on the light energy density.

FIG. 8 is a graph showing the scavenging of reactive oxygen species by the reactive oxygen species-scavenging drug.

FIG. 9 is a graph showing cell viability.

FIG. 10 is a graph showing cell proliferation.

FIG. 11 is a graph showing the wound enclosure through cell migration.

### DETAILED DESCRIPTION

Hereinafter, embodiments of the present disclosure are described in detail with reference to the accompanying drawings, and similar or identical elements are assigned the same reference numerals irrespective of figure numbers, and redundant descriptions thereof are omitted. In the following description of the embodiments, it will be understood that, when a layer (or film), a region, a pattern, or a structure is referred to as being “on” or “under” another substrate, another layer (or film), another region, another pad, or another pattern, it may be “directly” on the other substrate, layer (or film), region, pad, or pattern, or may be indirectly thereon with one or more intervening layers. Such a position of each layer will be described with reference to the drawings. In the drawings, the thickness or size of each layer is exaggerated, omitted, or schematically illustrated for convenience and clearness of description. In addition, the size of each element does not fully represent its actual size.

It should be understood that the terms “comprise”, “include”, “contain”, and the like herein specify certain features, numbers, steps, operations, elements, or some or combinations thereof, but do not preclude the presence or possibility of one or more other features, numbers, steps, operations, elements, or some or combinations thereof in addition to the description.

The terms first, second, and the like may be used herein to describe various elements. These elements should not be limited by these terms, as these terms are only used to distinguish one element from another unless stated otherwise or the context clearly indicates otherwise.

In the description of embodiments herein, a detailed description of known techniques associated with the present disclosure may be omitted to avoid unnecessarily obscuring the subject matter of the present disclosure.

The accompanying drawings are intended to facilitate the understanding of embodiments herein, and the technical spirit disclosed herein is not limited by the accompanying drawings, and rather should be construed as including all the modifications, equivalents and substitutes within the spirit and technical scope of the present disclosure.

The disclosures of cited papers and patent documents herein are entirely incorporated by reference into the present specification, and the level of the technical field within which the present disclosure falls and details of the present disclosure are explained more clearly.

Hereinafter, embodiments of the present disclosure will be described with reference to the accompanying drawings.

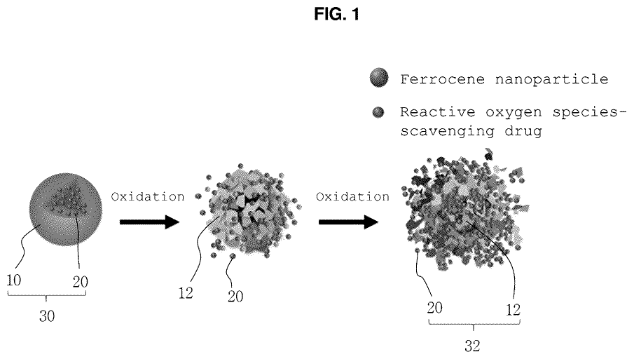

FIG. 1 shows reactive oxygen species-responsive drug delivery particle according to an embodiment of the present disclosure and the release of a drug from the reactive oxygen species-responsive drug delivery particle.

Photobiomodulation is a treatment method whereby a light with a predetermined wavelength is absorbed into cytochrome c oxidase in mitochondria within the cells, thereby promoting metabolic activity by adenosine triphosphate (ATP), reactive oxygen species (ROS), and nitric oxide (NO) generated within the cells. In an embodiment of the present disclosure, a red light with a wavelength of 600-700 nm may be used. Preferably, a red light with a wavelength of 630 nm may be used.

Reactive oxygen species are not only byproducts of photobiomodulation, but also serve as a secondary messenger in the cellular metabolism. The second messenger is a small, water-soluble substance involved in the signaling pathways of organisms, wherein when cells receive an external signal, the second messenger is generated to secondarily transmit and amplify the signal internally. The presence of an appropriate amount or concentration of reactive oxygen species in cells mediates intracellular signaling, defends against pathogens, and promotes cellular metabolic activity and extracellular matrix formation. However, excessive reactive oxygen species may disrupt the balance of the wound healing system to cause oxidative stress, DNA mutation, and cell structure damage, resulting in chronic wounds or scars. Therefore, the amount or concentration of reactive oxygen species needs to be adjusted to an appropriate level for effective wound healing.

A drug delivery particle 30 according to an embodiment of the present disclosure may be composed of a particle having responsiveness to reactive oxygen species. The reactive oxygen species-responsive drug delivery particle 30 may be composed of a ferrocene particle 10 containing a polymer containing ferrocene. The ferrocene particle 10 may preferably be a ferrocene nanoparticle. The size of the ferrocene nanoparticle 10 may have a diameter of 20 to 200 nm. The polydispersity index (PDI) of the ferrocene nanoparticles may be 0.2 or less. The surface charge of the ferrocene nanoparticle may be −5 mV.

The polymer containing ferrocene may be, for example, ferrocenylmethyl methacrylate (FMMA). Specifically, the ferrocene nanoparticle 10 may be formed of ferrocenylmethyl methacrylate (FMMA), polyethylene glycol methacrylate (PEGMA), and methacrylic acid (MAA). Polyethylene glycol methacrylate may be bound to ferrocenylmethyl methacrylate, and methacrylic acid may be bound to polyethylene glycol methacrylate. The molar ratio of ferrocenylmethyl methacrylate, polyethylene glycol methacrylate, and methacrylic acid may be 1:1:1 to 1:5:10.

The ferrocene nanoparticle 10 according to an embodiment of the present disclosure may be manufactured as follows.

After 0.4 mmol ferrocenylmethyl methacrylate (FMMA), 1 mmol polyethylene glycol methacrylate (PEGMA), 1 mmol methacrylic acid (MAA, 99%), and 0.12 mmol AIBN as a radical initiator are dissolved in 10 ml of anhydrous tetrahydrofuran (THF), the mixture is stirred at 70° C. for 24 hours to perform a polymerization reaction.

### MAA:

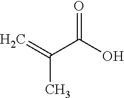

### PEGMA:

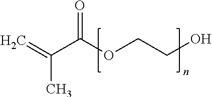

(where, n is a natural number of 1 or greater)

### FMMA:

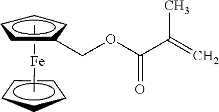

The ferrocene polymer prepared by the radical polymerization reaction is dissolved in THF to generate a ferrocene precursor solution and produce ferrocene nanoparticles 10 of Chemical Formula 1.

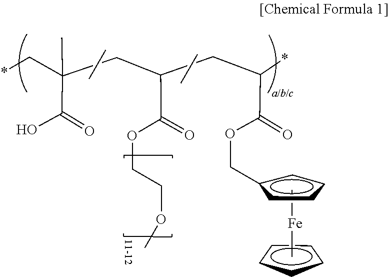

The ferrocene nanoparticle 10 has responsiveness to reactive oxygen species, and is used as a carrier for a drug treatment system by loading a drug therein. A reactive oxygen species-scavenging drug 20 may be loaded in the ferrocene nanoparticle 10. The reactive oxygen species-scavenging drug 20 may respond to reactive oxygen species generated during photobiomodulation, thereby serving to scavenge some of excessively generated reactive oxygen species. In an embodiment of the present disclosure, the reactive oxygen species-scavenging drug may contain a Centella asiatica component. Centella asiatica has an antioxidant effect and thus can prevent cell damage and scavenge reactive oxygen species.

Centella asiatica may be contained in an amount of 10 to 40 wt %, preferably 10 to 20 wt %, and more preferably 20 wt %, based on the weight of the ferrocene polymer.

In an environment of a high amount or concentration of reactive oxygen species, the size of the ferrocene nanoparticle 10 may increase, resulting in the rupture of the ferrocene nanoparticle 10, thereby releasing the reactive oxygen species-scavenging drug 20 loaded therein. As shown in FIG. 1, the ferrocene nanoparticle 10 is oxidized by reactive oxygen species, causing an increase in size and subsequently rupture, which leads to the release of the reactive oxygen species-scavenging drug 20 from the ruptured particle 12. In FIG. 1, the reference numeral 32 denotes a reactive oxygen species-responsive drug delivery particle, of which the reactive oxygen species-scavenging drug 20 is released by rupture of the ferrocene nanoparticle 10.

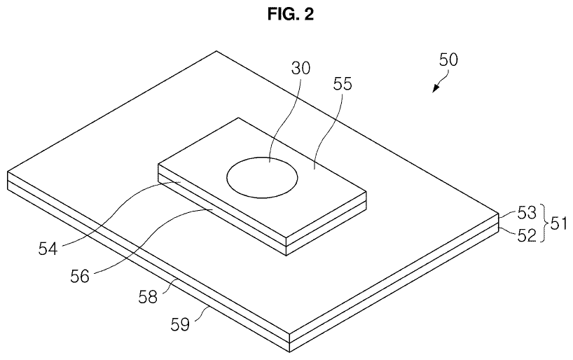

FIG. 2 is a perspective diagram schematically showing a device for wound healing according to an embodiment of the present disclosure.

A device for wound healing 50 according to an embodiment of the present disclosure includes a band part 51, a light source part 54, a power supply part 56, and reactive oxygen species-responsive drug delivery particles 30. The device for wound healing 50 may be in the form of a patch that is attached to the skin.

The light source part 54 emits light for the phototherapy of the human skin. The light source part 54 may be composed of a laser, an LED, an organic light-emitting diode (OLED), or the like. Since the laser and LED are point light sources, the target wound site may experience high temperatures due to the condensation of light energy or may be non-uniformly treated. Therefore, it is preferable to use an organic light-emitting diode capable of uniformly emitting light in the form of a surface light source.

The organic light-emitting diodes used in the light source part 54 may be composed of a substrate, a lower electrode disposed above the substrate, an OLED organic material layer disposed above the lower electrode, and an upper electrode disposed above the OLED organic material layer. The substrate, upper and lower electrodes, and OLED organic material layer constituting the organic light-emitting diode may all be composed of flexible materials. As described above, the light source part 54 may emit a red light with a wavelength of, for example, 600 to 700 nm, and preferably 630 nm.

The power supply part 56 includes a battery and is electrically connected to the light source part 54 to supply current to the light source part 54 to enable the light source part 54 to emit light. The power supply unit 56 may also be constructed of a flexible material. In FIG. 2, the light source part 54 and the power supply part 56 are disposed in a stacked structure, but the structure of the light source part 54 and the power supply part 56 is not limited thereto.

The light source part 54 and the power supply part 56 according to an embodiment of the present disclosure may employ the structures disclosed in the Korea Patent Publication Nos. 10-1906181 and 10-1792659 by the present applicant.

The band part 51 is composed of a support 52 and an adhesive layer 53. The support 52 may be formed as a thin rectangular flat plate with a first surface 58 and a second surface 59 so that the support can be attached to the human skin. A plastic film may be used as a material of the support 52. Examples of the plastic used for the support 52 may be polyolefin, polyethylene, polypropylene, a polyester, a polystyrene, a polyamide, a polyvinyl alcohol, an ethylene-vinyl acetate copolymer, a polyurethane, a styrene-isoprene-styrene copolymer, a styrene-butadiene-styrene copolymer, or a styrene-ethylene-propylene-styrene copolymer, which may be used alone or in combination.

The adhesive layer 53 may be formed on the first surface 58 of the support. The adhesive layer 53 may be formed of a pressure-sensitive adhesive. The adhesive layer 53 may be formed of a pressure-sensitive adhesive causing low irritation to the skin, and examples thereof may include an acrylic pressure-sensitive adhesive, a natural rubber-based pressure-sensitive adhesive, a synthetic rubber-based pressure-sensitive adhesive, a silicone pressure-sensitive adhesive, a vinyl ester-based pressure-sensitive adhesive, a vinyl ether pressure-sensitive adhesive, a urethane pressure-sensitive adhesive, and the like.

The light source part 54, the power supply part 56, and the reactive oxygen species-responsive drug delivery particles 30 are disposed above the first surface 58 of the band part. For example, the light source part 54 and the power supply part 56 may be disposed on the adhesive layer 53 of the band part. The active oxygen species-responsive drug delivery particles 30 may be applied onto a light-emitting surface 55 of the light source part or around the light-emitting surface 55. The active oxygen species-responsive drug delivery particles 30 may be applied above the first surface 58 of the band part at a separate region outside the light source part 54. The device for wound healing 50 is attached to the skin in a direction in which the adhesive layer 53 is in close contact with the skin.

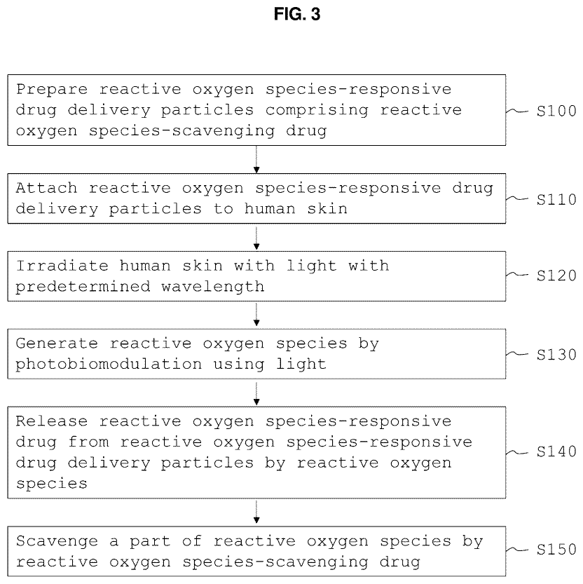

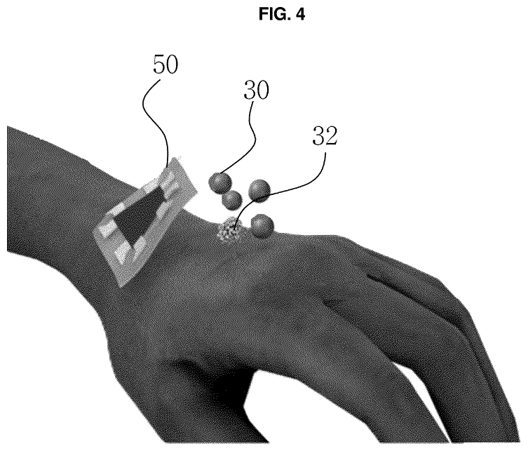

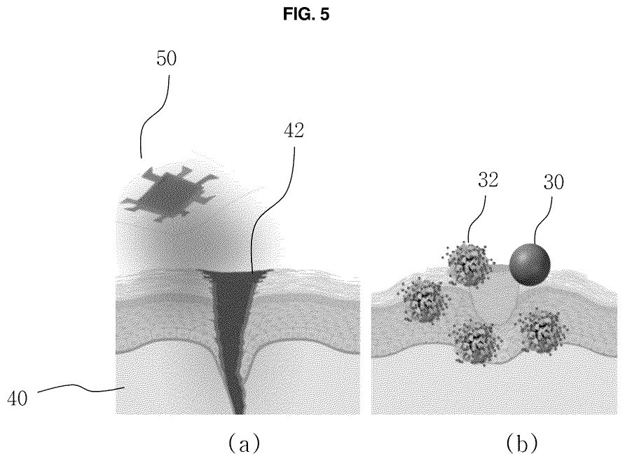

FIG. 3 is a flow chart of a treatment method according to an embodiment of the present disclosure; FIG. 4 schematically shows the treatment method according to the embodiment of the present disclosure; and FIG. 5 shows a treatment procedure by the treatment method according to the embodiment of the present disclosure.

The wound healing method according to the embodiment of the present disclosure may include: preparing reactive oxygen species-responsive drug delivery particles containing a reactive oxygen species-scavenging drug (S100); attaching the reactive oxygen species-responsive drug delivery particles to the human skin (S110); irradiating the human skin with a light with a predetermined wavelength (S120); generating reactive oxygen species by photobiomodulation using the light (S130); releasing the reactive oxygen species-responsive drug from the reactive oxygen species-responsive drug delivery particles by the reactive oxygen species (S140); and scavenging some of the reactive oxygen species by the reactive oxygen species-scavenging drug (S150).

The wound healing method will be specifically described as follows.

The device for wound healing 50 according to an embodiment of the present disclosure includes reactive oxygen species reactive drug delivery particles 30 having a reactive oxygen species-scavenging drug 20. As shown in FIGS. 4 and 5A, the device for wound healing 50 is attached to a wound site 42 of the skin 40, thereby allowing the reactive oxygen species-responsive drug delivery particles 30 to be attached to the wound site 42 of the human skin 40. The reactive oxygen species-responsive drug delivery particles 30 attached to the wound site 42 are absorbed into the skin.

When the light emitted from the light source part 54 of the device for wound healing 50 is applied to the skin 40, photobiomodulation is initiated, and during the photobiomodulation, reactive oxygen species as a secondary messenger are generated. As shown in FIG. 5B, the reactive oxygen species-responsive drug delivery particles 30 absorbed into the skin respond to the reactive oxygen species, resulting in the rupture of ferrocene nanoparticles 10, thereby releasing the reactive oxygen species-scavenging drug 20, for example, Centella asiatica, loaded inside the ferrocene nanoparticles 10. Centella asiatica may react with excessive reactive oxygen species within cells in the wound site 42 to scavenge some of the generated reactive oxygen species.

The reactive oxygen species generated by photobiomodulation have a wound healing effect. However, excessively generated reactive oxygen species may disturb the balance of the wound healing system of the human body, causing oxidative stress, DNA mutation, and cell structure damage. Therefore, the reactive oxygen species in the wound site 42 may be adjusted to an appropriate concentration for wound healing by the reactive oxygen species-scavenging drug 20, such as Centella asiatica, released from the ferrocene nanoparticles 10 in response to the reactive oxygen species.

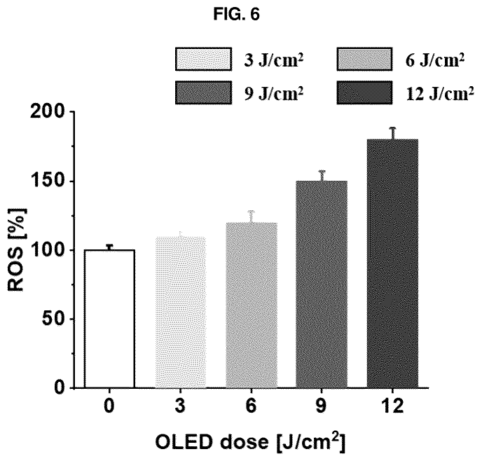

FIG. 6 is a graph showing the generation of reactive oxygen species depending on the light energy density.

As shown in FIG. 6, the amount of reactive oxygen species generated by photobiomodulation increased in proportion to the energy density of the applied light. In FIG. 6, the x-axis represents the energy density (J/cm²) of the irradiated light, and the y-axis represents the relative amount (%) of reactive oxygen species relative to a control group (sample not irradiated with light, i.e., 0 J/cm²).

Untreated NIH 3T3 cells were irradiated with a red light of 630 nm at 3, 6, 9, and 12 J/cm², and the amount of reactive oxygen species generated for each condition was measured. Compared with the control group that was not irradiated with the light, the amounts of reactive oxygen species generated in the cells irradiated with the light was increased by 10%, 20%, 50%, and 80%, respectively.

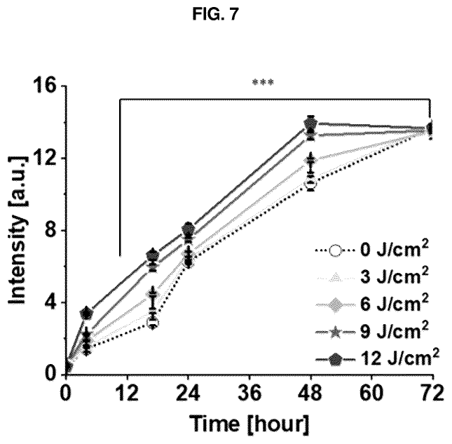

FIG. 7 is a graph showing the drug release rate depending on the light energy density.

To measure the drug release rate from the ferrocene nanoparticles 10 depending on the responsiveness to reactive oxygen species, Nile red, which exhibits unique hydrophobic fluorescence, was selected as a model drug. As the amount of reactive oxygen species generated increased with the increased energy density of the irradiated light, the release amount of Nile red loaded in the ferrocene nanoparticles increased. In FIG. 7, the x-axis represents time (hour), and the y-axis represents the intensity (a.u.) of fluorescence according to the amount of Nile red released.

Since the intensity of fluorescence increased according to the amount of released Nile red, the release amount of drug was calculated based on the measured intensity of fluorescence. When irradiated with a red light of 630 nm, the Nile red loaded in the ferrocene nanoparticles 10 was promptly released compared with the control group (when the irradiated light was 0 J/cm².) It can be seen that the drug release rate from the ferrocene nanoparticles 10 increases as the energy density of the irradiated light increases. That is, as the energy density of light increases, the reactive oxygen species increases, leading to an increase in the reaction between the ferrocene nanoparticles and the reactive oxygen species, thereby attaining prompt drug release.

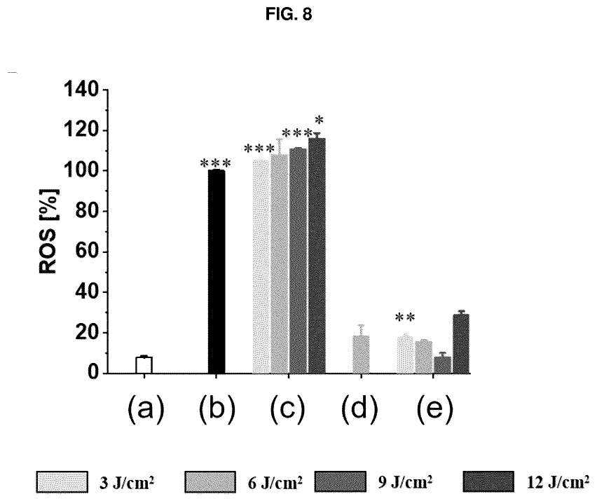

FIG. 8 is a graph showing the scavenging of reactive oxygen species by the reactive oxygen species-scavenging drug.

In FIG. 8, (a) shows untreated NIH 3T3 cells, with the amount or concentration of reactive oxygen species measured to be 8% corresponding a value in normal cells. In FIG. 8, (b) shows an environment with 100% reactive oxygen species, which was induced by treating NIH 3T3 cells with hydrogen peroxide (H₂O₂) to artificially generate reactive oxygen species.

The cells in (b) in FIG. 8 were irradiated with a red light of 630 nm, with the light output density fixed at 5 mW/cm², and light energy densities of 3, 6, 9, and 12 J/cm². As shown in (c) in FIG. 8, as the light energy density increased, the amount of reactive oxygen species increased from 100% ((b) in FIGS. 8) to 105, 108, 110, and 116%, respectively. Compared with 100% of reactive oxygen species in the cells treated with hydrogen peroxide ((b) in FIG. 8), the amount of reactive oxygen species in NIH 3T3 cells irradiated with light slightly increased, in proportion to the light irradiation time.

In FIG. 8, (d) shows that the amount of reactive oxygen species within the cell was reduced from 100% to 18.3% by applying a reactive oxygen species-responsive drug delivery particles 30 to the cells of (b) in FIG. 8.

In FIG. 8, (e) shows the amounts of reactive oxygen species generated when the reactive oxygen species-responsive drug delivery particles 30 were applied to the cells of (b), which were then irradiated at light energy densities of 3, 6, 9, and 12 J/cm², respectively. When reactive oxygen species-responsive drug particles 30 were applied to an environment where reactive oxygen species temporarily increased by light irradiation, the amount of reactive oxygen species was reduced from 105, 108, 110, and 116% ((c) in FIGS. 8) to 18, 16, 8, and 29% ((e) in FIG. 8), respectively.

In an environment similar to a wound environment, photobiomodulation generated reactive oxygen species, which increased the release rate of the reactive oxygen species-responsive drug 20, thereby enhancing the effect of scavenging some of the excessive reactive oxygen species. When the energy density of the irradiated light was 3-12 J/cm², preferably 3-11 J/cm², and more preferably 3-9 J/cm², the reactive oxygen species generated by light irradiation effectively react with the reactive oxygen species-responsive drug delivery particles 30, thereby scavenging the reactive oxygen species more promptly and effectively.

The reactive oxygen species reached a smallest value of 8% when the energy density of the irradiated light was 9 J/cm². When the energy density of the irradiated light exceeds 12 J/cm², the amount of reactive oxygen species generated by photobiomodulation surpassed the reactive oxygen species scavenging efficacy of the reactive oxygen species-scavenging drug 20, thus lowering the drug efficacy of reducing reactive oxygen species. The reactive oxygen species generated by photobiomodulation can increase the release amount of the reactive oxygen species-scavenging drug 20 loaded in the ferrocene nanoparticles 10, thereby regulating the amount of reactive oxygen species within cells in the wound site to be similar to that in normal skin.

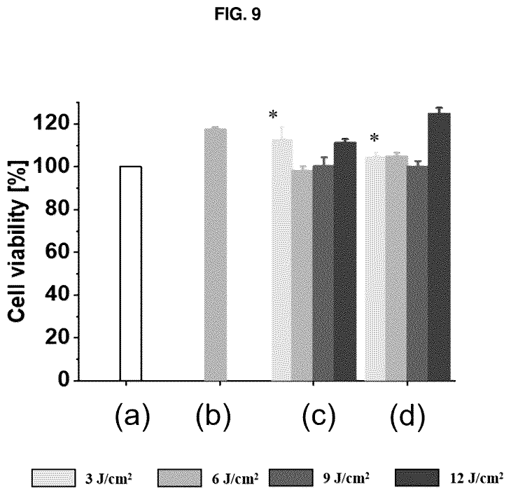

FIG. 9 is a graph showing cell viability.

The cytotoxicity was evaluated to investigate biological safety, and the cytotoxicity was evaluated by measuring cell viability 24 hours after light and drug treatment. The cell viability means the number of healthy living cells in a sample, and a cell viability of 70% or more may be determined to be non-toxic according to ISO standards.

In FIG. 9, (a) shows untreated NIH 3T3 cells, (b) shows the cells of (a) receiving the reactive oxygen species-responsive drug delivery particles 30, (c) shows the cells of (a) irradiated with a red light of 630 nm, at the light output density fixed at 5 mW/cm², with light energy densities of 3, 6, 9, and 12 J/cm², respectively, and (d) shows the cells of (a) receiving the reactive oxygen species-responsive drug delivery particles 30 and irradiated at light energy densities of 3, 6, 9, and 12 J/cm², respectively.

Compared with the control group ((a) of FIG. 9), (d) in FIG. 9 showed no reduction in cell viability, regardless of the concentration of the active oxygen species-scavenging drug 20 and the light irradiation time, and thus no toxic effect was observed in NIH 3T3 cells. It can be therefore identified that the combined therapy of light irradiation and reactive oxygen species-responsive drug delivery particles 30 according to embodiments of the present disclosure satisfies biological safety.

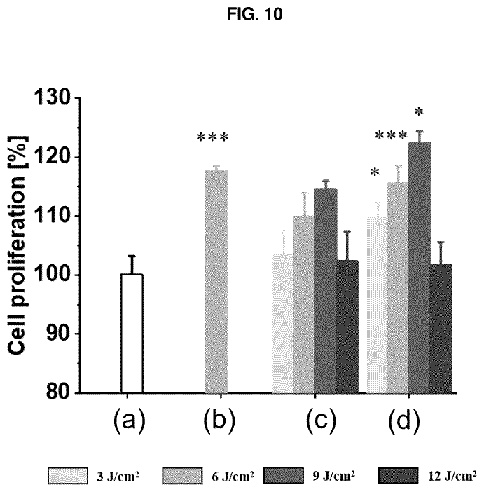

FIG. 10 is a graph showing cell proliferation.

The cell proliferation was investigated after the application of red light irradiation and reactive oxygen species-responsive drug delivery particles 30. (a), (b), (c), and (d) in FIG. 10 correspond to (a), (b), (c), and (d) in FIG. 9, respectively.

It was investigated whether the cell proliferation depends on the energy density of the irradiated light. As shown in (c) in FIG. 10, an energy density of 3-9 J/cm² of the irradiated light was effective for cell proliferation, and in particular, the cell proliferation at 9 J/cm² was improved by 14.6% compared with the control group (a). The cell proliferation effect at 12 J/cm² was not observed compared with the control group (a), and the reason is that the amount of reactive oxygen species generated increased by light irradiation at 12 J/cm². The application of the reactive oxygen species-responsive delivery particles 30 showed an 17.8% increase in cell proliferation compared with the control group ((b) in FIG. 10).

As shown in (d) in FIG. 10, under the combined application of light irradiation and the reactive oxygen species-responsive drug delivery particles 30, the cell proliferation increased in proportion to the energy density of the irradiated light, resulting in a 22.3% increase in cell proliferation at 9 J/cm² compared with the control group.

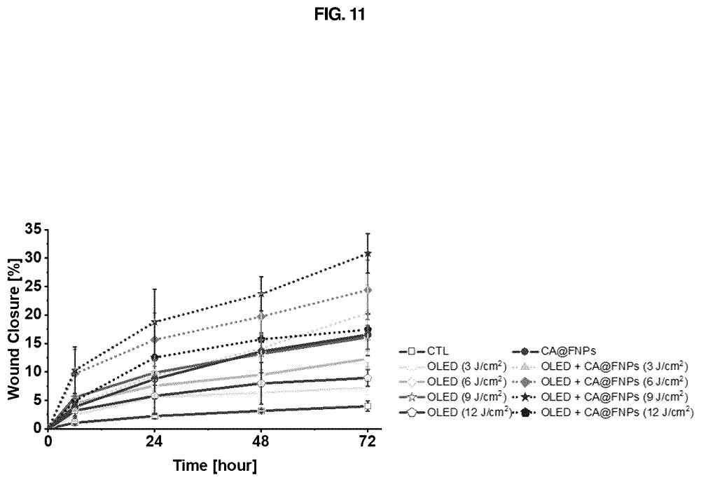

FIG. 11 is a graph showing the wound enclosure through cell migration.

Cell migration refers to the movement of cells toward a damaged area to repair a wound caused by physical damage. The wound healing assay is a research technique for observing cell migration on a two-dimensional plane in vitro, which involves making mechanical scratches on the surface of a cell confluent monolayer using a sharp tool. After the combined application of light irradiation and reactive oxygen species-responsive drug delivery particles 30, the cell migration was evaluated through the degree of scratch reduction for 72 hours.

In FIG. 11, CTL denotes the control group, CA@FNPs denotes the application of reactive oxygen species-responsive drug delivery particles 30 alone, OLED denotes the irradiation of a red light of 630 nm alone, and OLED+CA@FNPs denotes the combined application of a red light of 630 nm and reactive oxygen species-responsive drug delivery particles 30.

The experiment was conducted with the energy density of the irradiated light being 3-12 J/cm². At the energy density of 3-9 J/cm² of the irradiated light, the cell migration was improved as the energy of the irradiated light increased, and at 12 J/cm², the cell migration was again reduced. For 72 hours, the scratches on the cells were reduced by 19.1% and 17% through the application of CA@FNPs and OLED alone, respectively, and reduced by 34.36% through OLED+CA@FNPs. Therefore, the combined therapy of light irradiation and reactive oxygen species-responsive drug delivery particles 30 according to embodiments of the present disclosure can improve cell migration through reactive oxygen species regulation capacity.

The reactive oxygen species generated by photobiomodulation ruptured ferrocene nanoparticles up to 2.3 times faster than the control group within 17 hours. The reactive oxygen species generated by red light irradiation can promote the release of the reactive oxygen species-scavenging drug 20 from the reactive oxygen species-responsive drug delivery particles 30, leading to an enhanced reactive oxygen species scavenging effect. The reactive oxygen species-responsive drug delivery particles 30 can reduce reactive oxygen species by 102%, thereby enhancing drug efficacy by 124% compared with the control group. Furthermore, the red light treatment and the reactive oxygen species-scavenging drug treatment showed excellent cell proliferation and migration effects in in vitro tests. The embodiments of the present disclosure enable cell proliferation improved by 22.38% compared with the control group. Furthermore, the embodiments of the present disclosure improved cell migration by 30.82% compared with the control group.

The specified matters and limited embodiments and drawings such as specific elements in the present disclosure have been disclosed for broader understanding of the present disclosure, but the present disclosure is not limited to the embodiments, and various modifications and changes are possible by those skilled in the art without departing from an essential characteristic of the present disclosure. The spirit of the present disclosure is defined by the appended claims rather than by the described embodiments above, and all changes and modifications that fall within metes and bounds of the claims, or equivalents of such metes and bounds are therefore intended to be embraced by the range of the spirit of the present disclosure. Additionally, the respective embodiments may be combined and operated together as needed.

What is claimed is:

1. Reactive oxygen species-responsive drug delivery particles comprising:
a ferrocene particle comprising a polymer containing ferrocene; and
a reactive oxygen species-scavenging drug loaded in the ferrocene particle.

2. The reactive oxygen species-responsive drug delivery particles of claim 1, wherein the ferrocene particle comprises ferrocenylmethyl methacrylate, polyethylene glycol methacrylate bound to the ferrocenylmethyl methacrylate, and methacrylic acid bound to the polyethylene glycol methacrylate and wherein the ferrocenylmethyl methacrylate, the polyethylene glycol methacrylate, and the methacrylic acid have a molar ratio of 1:1:1 to 1:5:10.

3. The reactive oxygen species-responsive drug delivery particle of claim 1, wherein the ferrocene particle is represented by Chemical Formula 1 below:

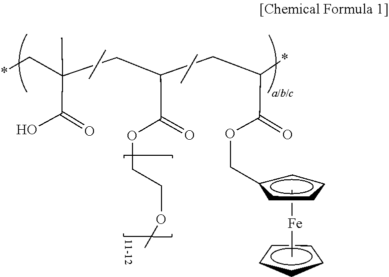

4. The reactive oxygen species-responsive drug delivery particle of claim 1, wherein the reactive oxygen species-scavenging drug comprises a Centella asiatica component.

5. The reactive oxygen species-responsive drug delivery particle of claim 2, wherein the ferrocene particle releases the reactive oxygen species-scavenging drug in response to a light with a wavelength of 600 to 700 nm.

6. A wound healing method, comprising:
preparing reactive oxygen species-responsive drug delivery particles comprising a reactive oxygen species-scavenging drug;
attaching the reactive oxygen species-responsive drug delivery particles to human skin;
irradiating the human skin with a light with a predetermined wavelength;
generating reactive oxygen species by photobiomodulation using the light;
releasing the reactive oxygen species-responsive drug loaded in the reactive oxygen species-responsive drug delivery particles by the reactive oxygen species; and
scavenging a part of the reactive oxygen species by the reactive oxygen species-scavenging drug.

7. The wound healing method of claim 6, wherein the reactive oxygen species-responsive drug delivery particles comprise a ferrocene particle comprising ferrocenylmethyl methacrylate, polyethylene glycol methacrylate bound to the ferrocenylmethyl methacrylate, and methacrylic acid bound to the polyethylene glycol methacrylate and a reactive oxygen species-scavenging drug loaded in the ferrocene particle
and wherein the ferrocenylmethyl methacrylate, the ferrocenylmethyl methacrylate, and the polyethylene glycol methacrylate have a molar ratio of 1:1:1 to 1:5:10.

8. The wound healing method of claim 7, wherein the ferrocene particle is represented by Chemical Formula 1 below:

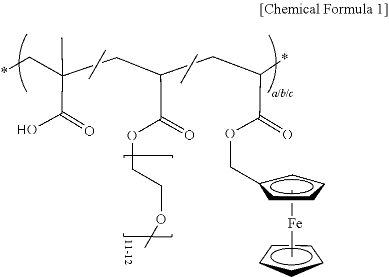

9. The wound healing method of claim 7, wherein the reactive oxygen species-scavenging drug comprises a Centella asiatica component.

10. The wound healing method of claim 7, wherein the ferrocene particle releases the reactive oxygen species-scavenging drug in response to a light with a wavelength of 600 to 700 nm.

11. A device for wound healing, comprising:
a band part comprising a first surface with an adhesive layer formed thereon;
a light source part disposed above the first surface to emit a light with a predetermined wavelength;
a power supply part disposed above the first surface and electrically connected to the light source part; and
reactive oxygen species-responsive drug delivery particles disposed above the first surface,
wherein the reactive oxygen species-responsive drug delivery particles comprise:
a ferrocene particle comprising a polymer containing ferrocene; and
a reactive oxygen species-scavenging drug loaded in the ferrocene particle.

12. The device of claim 11, wherein the ferrocene particle comprises ferrocenylmethyl methacrylate, polyethylene glycol methacrylate bound to the ferrocenylmethyl methacrylate, and methacrylic acid bound to the polyethylene glycol methacrylate and wherein the ferrocenylmethyl methacrylate, the polyethylene glycol methacrylate, and the methacrylic acid have a molar ratio of 1:1:1 to 1:5:10.

13. The device of claim 11, wherein the ferrocene particle is represented by Chemical Formula 1 below:

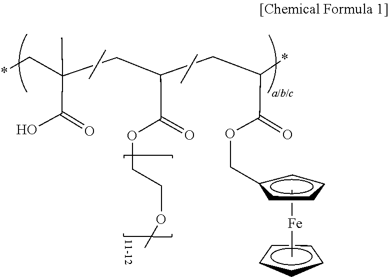

14. The device of claim 11, wherein the reactive oxygen species-scavenging drug comprises a Centella asiatica component.

15. The device of claim 11, wherein the reactive oxygen species-responsive drug delivery particles are applied onto an emitting surface of the light source part.

16. The device of claim 12, wherein the ferrocene particle releases the reactive oxygen species-scavenging drug in response to a light with a wavelength of 600 to 700 nm.
